(19) United States
(12) Patent Application Publication
(10) Pub. No.: US 2004/0234611 A1
(43) Pub. Date: Nov. 25, 2004

(54) Ophthalmic deport formulations for periocular or subconjunctival administration

(76) Inventors: Marklus Ahlheim, Staufen (DE); Michael Ausborn, Lorrrach (DE); David Bodmer, Klingnau (CH); Christian Schoch, Muttenz (CH)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/489,752

(22) PCT Filed: Sep. 13, 2002
(86) PCT No.: PCT/EP02/10314

(30) Foreign Application Priority Data
Sep. 14, 2001 (GB) 0122318.9

(51) Int. Cl. A61K 9/14

(52) U.S. Cl. 424/489

(57) ABSTRACT

The present invention relates to ophthalmic depot formulations comprising an active agent, e.g. embedded in a pharmacologically acceptable biocompatible polymer or a lipid encapsulating agent, e.g. for periocular or subconjunctival administration.

The present invention relates to ophthalmic depot formulations for treatment of ocular diseases, in particular treatment of retinal and choroidal diseases.

Ocular diseases are difficult to treat as introduction of active agents into the eye and maintenance of therapeutically effective concentration thereof is difficult.

Oral administration of an active agent or parenteral administration of an active agent to a site other than the eye provides the active agent systemically. In order to achieve effective intraocular concentrations, systemic administration may necessitate administration of often unacceptably high levels of the active agent.

Injection of compositions comprising an active agent into the eye may be ineffective as the active agent may be washed out or is depleted from within the eye into the general circulation resulting in necessity for repeated administration, e.g. three injections in three to 42 days as described in U.S. Pat. No. 5,632,984.

Introduction of slow release compositions, i.e. implants, into the eye, e.g. into an anterior segment or posterior segment of an eye as described in U.S. Pat. No. 4,853,224, e.g. into the suprachoroidal space or pars plana of the eye as described in U.S. Pat. No. 5,164,188, or e.g. into a site extrinsic to the vitreous comprising a suprachoroidal space, an avascular region of an eye, or a surgically-induced avascular region as described in U.S. Pat. No. 5,824,072, by injection or surgical methods such as laser ablation, photocoagulation, cryotherapy, heat coagulation and the like is extremely painful and stressful for the patient. Implants may have to be removed when therapy is completed or no longer efficacious. Applicants have found that ophthalmic depot formulations comprising an active agent may be administered, periocularly, e.g. retrobulbarly or sub-tenonly, or subconjunctivally.

Accordingly in one aspect, the present invention provides an ophthalmic depot formulation, comprising an active agent e.g. for periocular, e.g. retrobulbar or sub-tenon, or subconjunctival administration.

Ophthalmic depot formulations such as micro- or nanoparticle (hereinafter called microparticle) formulations, comprising an active agent e.g. embedded in a biocompatible pharmacologically acceptable polymer e.g. in an encapsulating polymeric matrix, or embedded in a lipid encapsulating agent have been found to be particularly suitable. The ophthalmic depot formulation may also comprise microparticles of essentially pure active agent, e.g. microparticles consisting of the active agent.

These microparticles have a high contact surface.

In one aspect, the present invention provides an ophthalmic depot formulation comprising microparticles of essentially pure active agent.

The microparticles of essentially pure active agent, e.g. microparticles consisting of the active agent, may be in amorphous or crystalline form e.g. with a particle size of 1 to 200 microns.

In another aspect, the present invention provides an ophthalmic depot formulation such as microparticles comprising an active agent, e.g. embedded in a biocompatible pharmacologically acceptable polymer or a lipid encapsulating agent.

The depot formulations, e.g. in particular microparticle formulations, of the present invention are adapted to release all or substantially all the active material over an extended period of time, e.g. several weeks up to 6 months. The matrix, e.g. polymer or lipid matrix, if present, is adapted to degrade sufficiently to be transported from the site of administration within one to 6 months after release of all or substantially all the active agent.

The polymer matrix of polymeric microparticles may be a synthetic or natural polymer. The polymer may be either a biodegradable or non-biodegradable or a combination of biodegradable and non-biodegradable polymers, preferably biodegradable.

Suitable polymers include

(a) linear or branched polyesters which are linear chains radiating from a polyol moiety, e.g. glucose,

(b) polyesters such as D-, L- or racemic polylactic acid, polyglycolic acid, polyhydroxybutyric acid, polycaprolactone, polyalkylene oxalate, polyalkylene glycol esters of acids of the Kreb's cycle, e.g. citric acid cycle, and the like and combinations thereof,

(c) polymers of organic ethers, anhydrides, amides, and orthoesters

(d) copolymers of organic esters, ethers, anhydrides, amides, and orthoesters by themselves or in combination with other monomers,

(e) polyvinylalcohol.

The polymers may be cross-linked or non-cross-linked, usually not more than 5%, typically less than 1%.

The desired rate of degradation of polymers and the desired release profile for active agent may be varied depending on the kind of monomer, whether a homo- or a copolymer or whether a mixture of polymers is employed.

The preferred polymers of this invention are linear polyesters, and branched chain polyesters. The linear polyesters may be prepared from the a-hydroxy carboxylic acids, e.g. lactic acid and glycolic acid, by the condensation of the lactone dimers, see e.g. U.S. Pat. No. 3,773,919.

Linear polylactide-co-glycolides (PLG) which are preferably used conveniently have a molecular weight between 25,000 and 100,000 and a polydispersity M_{w}/Mₙ e.g. between 1.2 and 2.

The branched polyesters preferably used according to the invention may be prepared using polyhydroxy compounds e.g. polyol e.g. glucose or mannitol as the initiator. These esters of a polyol are known and described in GB 2,145,422 B. The polyol contains at least 3 hydroxy groups and has a molecular weight of up to 20,000, with at least 1, preferably at least 2, e.g. as a mean 3 of the hydroxy groups of the polyol being in the form of ester groups, which contain poly-lactide or co-poly-lactide chains. Typically 0.2% glucose is used to initiate polymerization. The branched polyesters (Glu-PLG) have a central glucose moiety having rays of linear polylactide chains, e.g. they have a star shaped structure. The preferred polyester chains in the linear and star polymer compounds preferably used according to the invention are copolymers of the alpha carboxylic acid moieties, lactic acid and glycolic acid, or of the lactone dimers. The molar ratios of lactide: glycolide is from about 75:25 to 25:75, e.g. 60:40 to 40:60, with from 55:45 to 45:55, e.g. 55:45 to 50:50 the most preferred.

The branched polyesters having a central glucose moiety having rays of linear polylactide chains (Glu-PLG) may be prepared by reacting a polyol with a lactide and preferably also a glycolide at an elevated temperature in the presence of a catalyst, which makes a ring opening polymerization feasible.

The branched polyesters having a central glucose moiety having rays of linear polylactide chains (Glu-PLG) preferably have an average molecular weight Mₙ in the range of from about 10,000 to 200,000, preferably 25,000 to 100,000, especially 35,000 to 60,000 and a polydispersity e.g. of from 1.7 to 3.0, e.g. 2.0 to 2.5. The intrinsic viscosities of star polymers of Mₙ 35,000 and Mₙ 60,000 are 0.36 respectively 0.51 dl/g in chloroform. A star polymer having a Mₙ 52,000 has a viscosity of 0.475 dl/g in chloroform.

Suitable lipid encapsulating agents for lipid microparticles include phosphatidyl compounds such as phosphatidyl choline (PC), phosphatidyl serine (PS), and phosphatidyl ethanolamine (PE), sphingolipids, cerebrosides, ganglosides, steroids, e.g. cholesterol, etc.

The terms microsphere, microcapsule and microparticle are considered to be interchange-able with respect to the invention, and denote the encapsulation of the active agent by the polymer, preferably with the active agent distributed throughout the polymer, which is then a matrix for the active agent. In that case preferably the terms microsphere or more generally microparticle are used.

The microparticles, e.g. microspheres or microcapsules, may have a diameter from a few submicrons to a few millimeters, e.g. from about 0.01 microns to about 2 mm, e.g. from about 0.1 microns to about 500 microns. For pharmaceutical micro-particles, diameters of at most about 250 microns, e.g. 10 to 200 microns, preferably 10 to 130 microns, more preferably 10 to 90 microns, even more preferably 10 to 60 microns, are strived for, e.g. in order to facilitate passage through an injection needle.

Typically, the active agent will be from about 1 to 80, more usually 10 to 75% by weight of the polymeric microparticles and from 1 to 20% by weight of the lipid microparticles.

In another aspect, the present invention provides a liquid formulation, comprising a pharmaceutical acceptable polymer and a dissolved or dispersed active agent. Upon injection, the polymer forms a depot at the injection site, e.g. by gelifying or precipitating.

The depot formulations, in particular microparticle formulations, according to the present invention are suitable for the incorporation of a large variety of water soluble or hydrophobic active agents.

Active agents of particular interest include

i) anti-glaucoma drugs, such as the beta-blockers, e.g. timolol maleate, betaxolol, carteolol and metipranolol; epinephrine and prodrugs; such as dipivefrin; carbonic anhydrase inhibitors; such as dorzolamide, brinzolamide, acetazolamide, dichlorphenamide and methazolamide; dopaminergics, prostaglandins, docosanoids, alpha2 agonists; angiotensin II antagonists; alpha1 antagonists; cannabinoids; endothelin antagonists;

ii) miotics, e.g. pilocarpine, acetylcholine chloride, isoflurophate, demecarium bromide, echothiophate iodide, phospholine iodide, carbachol, and physostigmine;

iii) drugs for treatment of macular degeneration, such as interferon, particularly α-interferon; transforming growth factor (TGF), e.g. TGF-β;

iv) anti-cataract and anti-proliferative diabetic retinopathy (PDR) drugs, such as aldose reductase inhibitors: e.g. tolrestat, or angiotensin-converting enzyme inhibitors, e.g. lisinopril, enalapril;

v) drugs for treatment of age-related exudative macular degeneration (AMD), e.g. ocular neovascular disease, such as staurosporines, phthalazine derivatives;

vi) anti-clotting agents, such as tissue plasminogen activator, urokinase, and streptokinase;

vii) drugs for treatment of ocular inflammatory diseases such as cortico-steroids; e.g. prednisolone, triamcinolone, dexamethasone, fluocinolone, cortisone, prednisolone, fluorometholone and the like, non-steroidal anti-inflammatory drugs, such as ketorolac tromethamine, diclofenac sodium, indomethacin, flurbiprofen sodium, and suprofen;

viii) antibiotics, such as loridine (cephaloridine), chloramphenicol, clindamycin, amikacin, gentamicin, tobramycin, methicillin, lincomycin, oxacillin, penicillin, amphotericin B, polymyxin B, cephalosporin family, ampicillin, bacitracin, carbenicillin, cephalothin, colistin, erythromycin, streptomycin, neomycin, sulfacetamide, vancomycin, silver nitrate, sulfisoxazole diolamine, quinolones, and tetracycline;

ix) anti-fungal or anti-viral agents, such as miconazole, ketoconazole, idoxuridine, tri-fluridine, vidarabine (adenine arabinoside), acyclovir (acycloguanosine), gancyclovir, foscarnet sodium, cidofovir, valacyclovir, famciclovirtrisulfapyrimidine-2, nystatin, flucytosine, natamycin, aromatic diamidines e.g. dihydroxystilbamidine and piperazine derivatives, e.g. diethylcarbamaine;

x) cycloplegics and mydriatic agents, such as atropine, cyclopentolate, scopolamine, homatropine tropicamide and phenylephrine;

xi) drugs for the treatment of ocular neurodegenerative diseases such as isopropyl unoprostone, glutamate receptor antagonists, e.g. memantine, caspase inhibitors, calcium antagonists, sodium channel blockers, NOS-2 inhibitors or neurotrophic factors, e.g. glial derived neurotrophic factor (GDNF) or ciliary neurotrophic factor (CNTF);

xii) peptide drugs such as calcitonin, lypressin or a somatostatin or analogues thereof,

xiii) anti-VEGF drugs;

xiv) phosphodiesterase inhibitors;

xv) antisense drugs such as fomivirsen sodium;

xvi) immunosuppressive agents; such as azathioprine, cyclosporin A, methotrexate, colchicine;

xvii)drugs for the treatment of ocular angiogenesis such as angiostatic steroids, PKC inhibitors, VEGF antagonists, COX2 inhibitors, ACE inhibitors or angiotensin II antagonists;

xviii) free radical scavengers, e.g. alpha tocopherol, carotenoids, sulfhydryl-containing compounds.

Preferably, active agents are drugs for treatment of the orbit region and ocular appendages, and for treatment of retinal and choroidal diseases comprising but not limited to age-related macular degeneration, diabetic retinopathy, glaucoma, inflammation, e.g. endophthalmitis, and bacterial, fungal or viral infections. Even more preferably, the active agent is a staurosporine of formula (I), a phthalazine of formula (II) or an ophthalmically acceptable salt thereof. Even more preferred are the staurosporine of formula (I) wherein R is benzoyl (hereinafter compound A), and the phthalazine of formula (II) wherein Z is 4-pyrididyl, X is imino, n is 0, and Y is 4-chlorophenyl (hereinafter compound B).

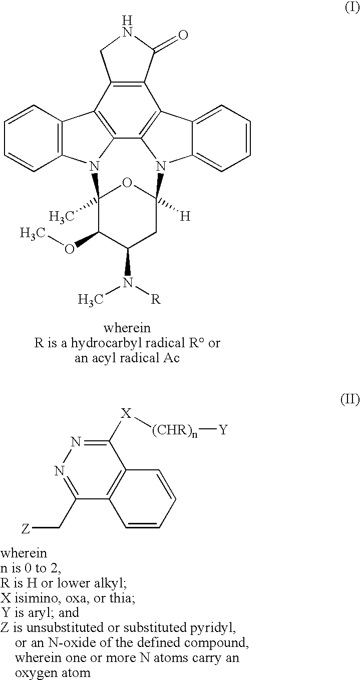

In another aspect, the present invention provides depot formulations and microparticles comprising a staurosporine of formula (I), a phthalazine of formula (II) or an ophthalmically acceptable salt thereof e.g. embedded in a biocompatible pharmacologically acceptable polymer, e.g. for periocular, e.g. retrobulbar or sub-tenon, or subconjunctival administration.

The microparticles of this invention may be prepared by any conventional technique, e.g. solvent evaporation, organic phase separation, spray drying, solvent extraction at low temperature or emulsion method, e.g. triple emulsion method. Using the phase separation or emulsion technique, the polymer is precipitated together with the drug, followed by hardening of the resulting product.

In another aspect, the present invention provides for a process for the production of microparticles comprising the steps of

a) dissolving the polymer or lipid encapsulating agent and the active agent in an organic solvent, e.g. methylene chloride,

b) mixing the solution of a) with an aqueous solution of polyvinyl alcohol (e.g. 0.5%). e.g. using a static mixer

c) collecting the generated microparticles, e.g. by a sedimentation, filtration or using a cyclon,

d) optionally washing of microparticles e.g. in a buffered solution of e.g. pH 3.0 to 8.0 or distilled water, and

e) drying under vacuo e.g. at a temperature of 20° C. to 40° C.

The invention also relates to the microparticles prepared by this process.

The microparticles and the depot formulations of the present invention are useful for treatment of the known ophthalmic indications of the particular active agent incorporated therein. The utility of the formulations of the present invention may be observed in standard animal trials and clinical trials.

In a further aspect, the present invention provides a method for treating an ocular disease which comprises:

i) providing a depot formulation, e.g. a microparticle formulation, comprising an active agent e.g. embedded in a pharmacologically acceptable biocompatible polymer or a lipid encapsulating agent, and

ii) administering said depot formulation, e.g. microparticle formulation, periocularly, e.g. retrobulbarly or sub-tenonly, or subconjunctivally.

This method permits diffusion of said active agent from said depot formulation, e.g. a microparticle formulation, to the site of said ocular disease, e.g. the choroid, optic nerve, retina or vitreous. Preferably, the active agent is maintained at an effective dosage for said ocular disease at the site of said ocular disease for an extended period of time, e.g. for several weeks up to 6 months.

The depot formulations, e.g. microparticle formulations, may be administered, periocularly, e.g. retrobulbarly or sub-tenonly, or subjconjunctivally in a variety of ways including injection, trocar etc. Preferably, the active agent particles or the microparticles are suspended in a suitable liquid carrier.

The exact amount of active agent embedded in the polymer, i.e. the exact amount of depot formulation, e.g. microparticles formulation, to be administered depends on a number of factors, e.g. the condition to be treated, the desired duration of treatment, the rate of release of active agent and the degradability of the polymeric matrix. The amount of active agent required may be determined on the basis of known in vitro or in vivo techniques. Repeated administration of the depot formulation of the invention may be effected when the polymeric matrix has sufficiently degraded.

Large amounts of active agent, e.g. up to 300 mg of active agent, e.g. in form of a suspension, may be administered in a single administration, e.g. in one injection. Frequency of dosing is variably dependent upon the severity of the syndrome. For severe cases dosing may occur once a month. The frequency is reduced when signs of the disease state show improvement. At that time dosing may be as infrequent as one dose every four or five months.

Filling may be effected before or after sterilization of the depot formulation. Sterilization of the formulation of the present invention and the primary package can be effected, e.g. by gamma irradiation e.g. at an energy of 25 kGy, without degradation of active agent and/or microparticles.

Following is a description by way of example only of depot formulations of this invention.

### EXAMPLE 1 to 3

### Preparations of microparticles

| | Ex. 1 | | Ex. 2 | | Ex. 3 | |
|---|---|---|---|---|---|---|
| compound A | 0.10 | g | 0.25 | g | 0.50 | g |
| Glu-PLG | 0.90 | g | 0.75 | g | 0.50 | g |
| methylene chloride | 2.5 | ml | 4.0 | ml | 9.5 | ml |
| 1.5% aq. polyvinyl alcohol | 500 | ml | 600 | ml | 900 | ml |
| 0.5% aq. polyvinyl alcohol | 3 | l | 3 | l | 3 | l |

Compound A and the polymer Glu-PLG are dissolved in the methylene chloride. The resulting solution is pumped through a static mixer together with a 1.5% solution of polyvinyl alcohol in water into a stirred solution of polyvinylalcohol in water (0.5%). The resulting suspension is heated to 42-48° C. with stirring within 60 min and kept at that temperature for further 30 min before the mixture is cooled down to about 22° C. within 50 min. The suspension is allowed to sediment for approximately 10 min. The aqueous solution of polyvinyl is reduced under vacuo. The microparticles are washed with water for approximately 5 min. After sedimentation for 10 min, the solution is removed and the microparticles are filtered through an Ultipor filter, washed with water and dried under vacuo.

1. An ophthalmic depot formulation comprising an active agent for periocular or subconjunctival administration.

2. A formulation according to claim 1 comprising of microparticles of essentially pure active agent.

3. A formulation according to claim 1 wherein the active agent is embedded in a biocompatible pharmacologically acceptable polymer or a lipid encapsulating agent.

4. A formulation according to claim 1 wherein the polymer is a poly-lactide-co-glycolide ester of a polyol.

5. A formulation according to claim 1 wherein the polymer is a 40/60 to 60/40 polylactide-co-glycolide ester of a polyol.

6. A formulation according to claim 1 comprising microparticles.

7. A formulation according to claim 6 wherein the external surface of the microparticles is substantially free of active agent.

8. A liquid formulation comprising a dissolved pharmaceutical acceptable polymer and a dissolved or dispersed active agent which formulation upon injection forms a depot at the injection site.

9. A formulation according to claim 1 wherein the active agent is present in an amount of up to 300 mg per dose for single administration.

10. A formulation according to claim 1 wherein the active agent is a staurosporine of formula (I), a phthalazine of formula (II) or an ophthalmically acceptable salt thereof.

11. A method for treating an ocular disease which comprises:
i) providing a depot formulation comprising an active agent, and
ii) introducing said depot formulation periocularly or subconjunctivally.

12. A method according to claim 11 wherein the active agent is embedded in a pharmacologically acceptable biocompatible polymer or a lipid encapsulating agent.

13. A method according to claim 11 wherein the active agent diffuses from said depot formulation to the site of said ocular disease.

14. A method according to claim 11 wherein the active agent is maintained at an effective dosage for said ocular disease at the site of said ocular disease for an extended period of time.

15. A method according to claim 11 wherein the active agent is maintained at an effective dosage for up to 3 months.

16. A microparticle comprising a staurosporine of formula (I), a phthalazine of formula (II) or an ophthalmically acceptable salt thereof embedded in a biocompatible pharmacologically acceptable polymer or a lipid encapsulating agent.
